(19) United States
(12) Patent Application Publication
(10) Pub. No.: US 2004/0081670 A1
(43) Pub. Date: Apr. 29, 2004

(54) Method for producing an active ingredient concentrate, and an active ingredient concentrate

(76) Inventor: Dariush Behnam, Rossdorf (DE)

Correspondence Address: JACOBSON HOLMAN PLLC, 400 SEVENTH STREET N.W., SUITE 600, WASHINGTON, DC, 20004, US

(21) Appl. No.: 10/470,749

(22) PCT Filed: Feb. 11, 2002
(86) PCT No.: PCT/EP02/01416

(30) Foreign Application Priority Data
Feb. 11, 2001 (DE) 101 09 708.5
Feb. 11, 2001 (DE) 101 08 614.8

(51) Int. Cl. A61K 9/00

(52) U.S. Cl. 424/400

(57) ABSTRACT

A method is described for the production of a water-soluble phase of an active substance, which is insoluble or only soluble with difficulty in water and is physiologically effective, the active substance, while heated, being stirred with an excess of a physiologically compatible solubiliser with an HLB value between 9 and 16 for the formation of a transparent intermediate product, the said intermediate product being cooled to room temperature. Also, a water-soluble concentrate of a physiologically active substance is described.

An active substance concentrate and a method of producing an active substance concentrate

The invention refers to a water-soluble concentrate which comprises a physiological active substance, which is insoluble or only soluble with difficulty in water, and a solubiliser, as well as a method for producing the concentrate.

Fat-soluble compounds, such as for example vitamin E, vitamin A and other carotenoids or also coenzyme Q₁₀, are absorbed in dependence of the presence of bile salts and pancreatic enzymes. A process of so-called micelle formation in the intestine precedes the absorption process; this is necessary so that the fat-soluble compounds can be “packed” and in this way the various barriers of the intestinal mucosa overcome.

If the secretion of the bile fluid or pancreatic enzymes is disturbed, then this results in a so-called maldigestion or malabsorption of fat-soluble compounds. The best example of this is the illness cystic fibrosis where, due to deficient generation of pancreatic enzymes, the resorption of fat-soluble compounds is possible only to a very slight extent.

The special features of the absorption of fat-soluble micronutrients also become apparent in that the ingestion always increases when fat is offered simultaneously. Fat promotes on one hand the release of bile acid and pancreatic enzymes and, on the other hand, the formation of micelles, which then contain the said fat-soluble micronutrients.

Once the fat-soluble compounds have been ingested by the intestinal cells, they are then available there in a free form, i.e. they are no longer bound to micellar constituents. In this free form they are then rendered “water-soluble” again in that they are integrated into the transporters formed within the intestinal cells (lipoproteins-chylomicrons) and then released via the large lymphatic channels into the blood.

In order to be able to accept lipophilic compounds, the organism must render them water-soluble in two steps. The first step occurs in the intestine through the formation of the micelles, out of which the substance is then released again in the intestinal cell, and the second step is the formation of lipoproteins for transport in the blood. Therefore lipophilic substances which have been rendered water-soluble (clear solutions), but not those which are just dispersed in the aqueous medium (turbid solutions), are more rapidly and more efficiently absorbed by the organism than the original lipophilic substance.

Only very little data is available on the bioavailability of lipophilic micronutrients that have been rendered water-soluble (clear solutions). A technique for testing the bioavailability of such compounds is provided by the so-called in-vitro dissolution methods. In this connection, it is found how far a compound dissolves in the aqueous compartment or how far it is released from a certain galenic formulation. From U.S. Pat. No. 6,048,566 it is known that Q₁₀ that has been rendered water-soluble, in contrast to Q₁₀ from oily solutions or dispersions (turbid solutions), is released by up to 100 percent. This signifies though that Q₁₀ applied in this way is already available in a higher concentration in free form in the intestinal lumen and does not need to be released first by micelle formation or by decomposition of the lipids surrounding the Q₁₀.

The object of the invention is therefore to improve the bioavailability of physiologically important substances, which are insoluble or only soluble with difficulty in water, such as ω-3-fatty acids, α-lipoic acid (thioctic acid), ubichinons (e.g. coenzyme Q₁₀), phytosterins and others, and to simplify technologically the industrial processing of these substances.

The object of the invention is also to use the smallest possible amount of solubilisers, in particular polysorbates, in the production of the concentrates—taking into account a tolerance range for ensuring complete, stable water solubility—so that the ADI values (ADI=Acceptable Daily Intake) for the polysorbates according to the JECFA (=Joint FAO/WHO Expert Committee on Food Additives) and SCG substances (SCG=Scientific Committee on Food (EU)) such as tocopherols (e.g. α-tocopherols), ω-3-fatty acids, α-lipoic acid (thioctic acid) and ubichinons (e.g. coenzyme Q₁₀) are clearly undercut.

This object is solved according to the invention in that the substance which is insoluble or only soluble with difficulty in water is added to an excess of heated physiologically compatible solubiliser with an HLB value between 6 and 19, especially Polysorbate 20 or Polysorbate 80, the mixture being stirred under the influence of heat until a clear, viscous intermediate product is produced and the intermediate product is then cooled to room temperature. If the active substance is to be provided in the aqueous phase, hot distilled water is added to the hot intermediate product in such an amount that corresponds to the desired concentration of active substance; it is then stirred to obtain homogeneity and the phase produced in this way is then cooled quickly to room temperature.

If the phase is to be free of water, a hot triglyceride, for example a light vegetable oil with a high linoleic acid content, and hot polysorbate are added to the intermediate product in such amounts so as to give the desired concentration of active substance and are stirred again under the influence of heat until the concentrate becomes clear. After cooling the active substance is present in a water-free phase which can however be dissolved in water as required. This is particularly suitable for administering the active substance in capsule form which in the long term tolerates only a very low water content.

Both the water-free and the aqueous phases are soluble in water or fat and/or oil.

As measurements using single-phase chromatography show, the active substance is present both in the aqueous and in the water-free phase in molecular aggregates enclosed by polysorbate, whereby the polysorbate envelope in each case exhibits a diameter of about 30 nm; the polysorbate envelope with enclosed molecular aggregate can be regarded as a micelle.

In the physiological field the micelle formation according to the invention results in a substantially improved bioavailability of the active substance. The active substance which is insoluble or only soluble with difficulty in water does not first need to be rendered ingestible for the intestinal lumen through interaction with bile secretions.

Embodiments of the invention are given in the subclaims and explained in the following without restricting the generality of the claimed invention. The enclosed drawing shows

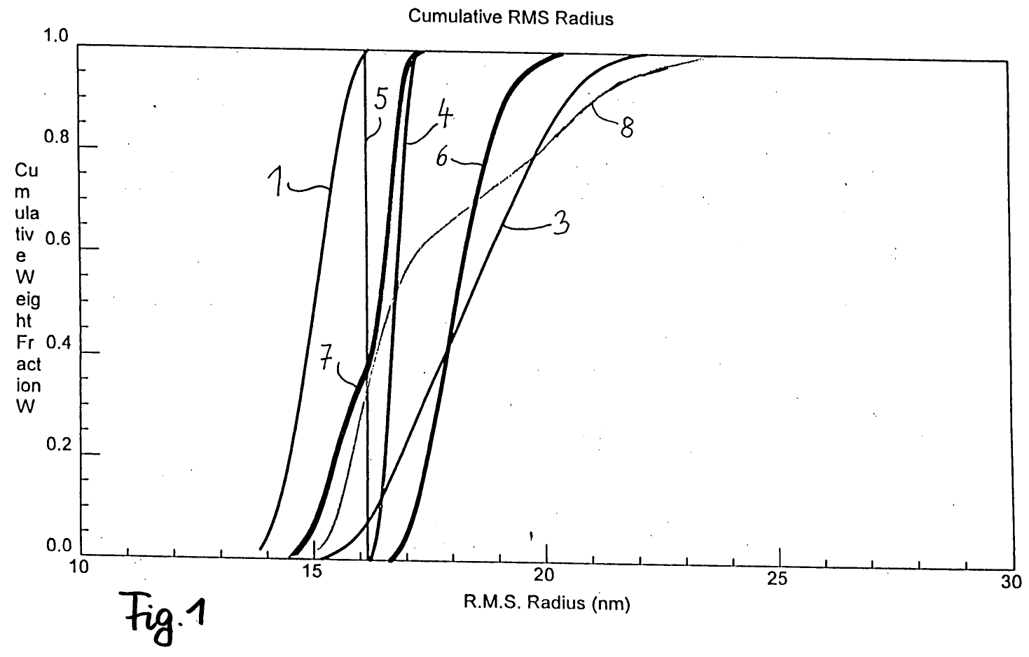

FIG. 1, a diagram of measured micelle radius distributions;

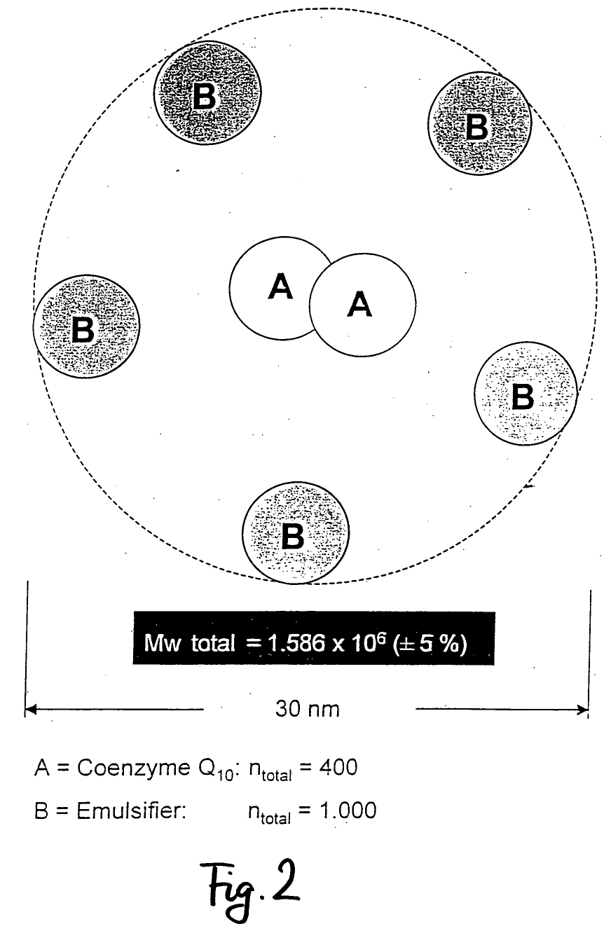

FIG. 2, a schematic explanation of active substance micelles with the coenzyme Q₁₀ as an example and

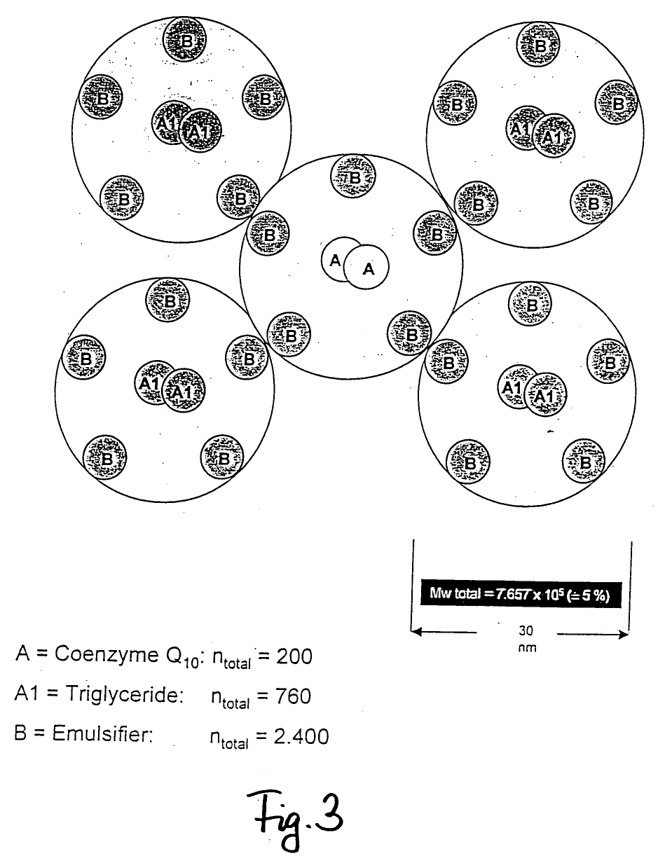

FIG. 3, a schematic explanation of the arrangement of micelles which contain an active substance (coenzyme Q₁₀) and an auxiliary substance (linoleic acid);

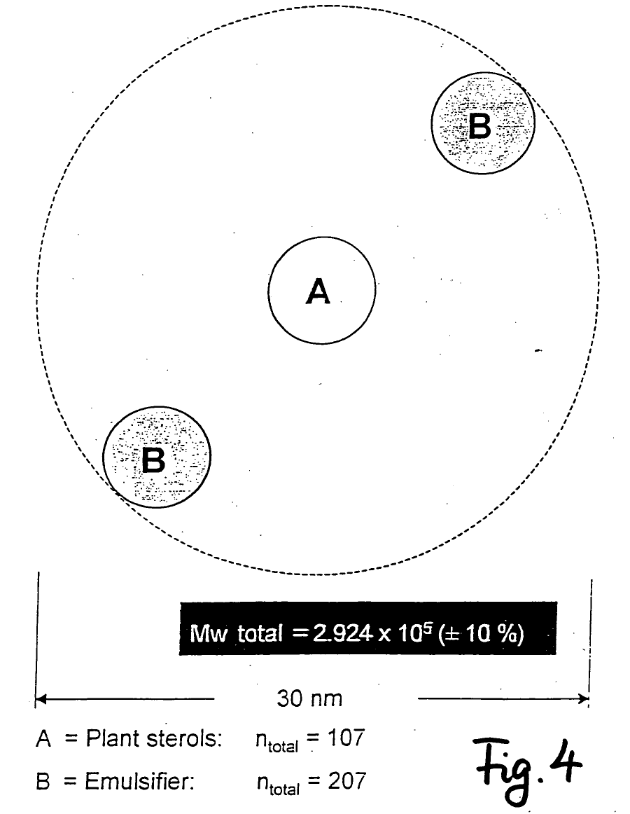

FIG. 4: a schematic representation of phytosterin micelles;

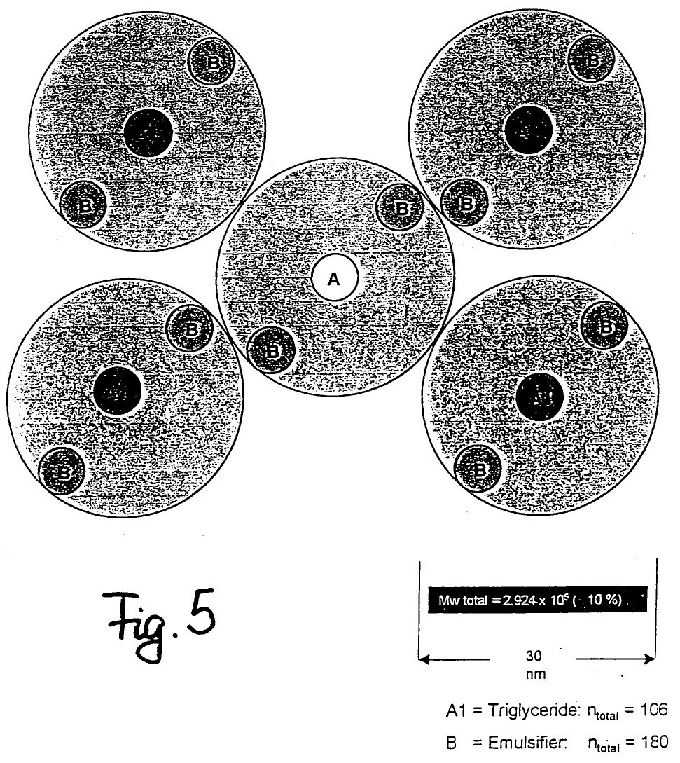

FIG. 5: a schematic representation of the distribution of additional linoleic acid micelles around a phytosterin micelle, and

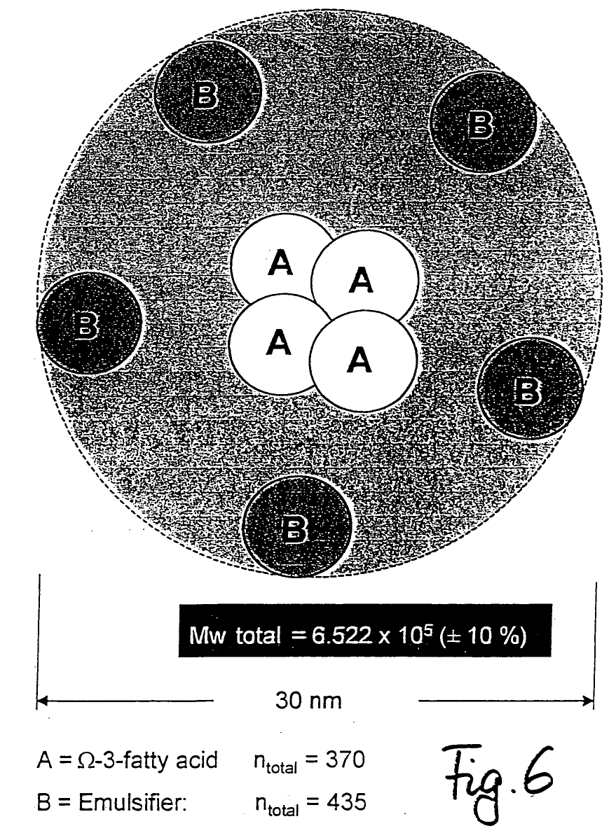

FIG. 6: a schematic representation of a micelle of an ω-3-fatty acid.

### EXAMPLE 1

### Coenzyme Q₁₀

In 77 parts by mass of Polysorbate 80 heated to 85° C., 23 parts by mass of coenzyme Q₁₀ are introduced and the mixture stirred for about 5 min. at 85° C. until a clear viscous mass of a slightly yellow colour is produced. The mass proportions of coenzyme Q₁₀ to Polysorbate 80 in the intermediate product are 1:3.35 and the ratio of the molecular numbers is 1:2.56, because the molecular weight of coenzyme Q₁₀ is 863.36 and that of Polysorbate 80 is 1130.00.

To obtain an aqueous phase of Q₁₀ from the intermediate product, which contains a Q₁₀ concentration of about 3%, approximately 865 parts by weight of hot water are added to the hot intermediate product and stirred under the influence of heat until a clear liquid is produced. Then the liquid is cooled quickly (for example, within about 1 to 2 minutes) to room temperature (about 20° C.) to give the finished aqueous phase of the coenzyme Q₁₀. The mean particle radius present in the phase was measured by field flow fractionation (FFF) with a DAWN EOS detector from Wyatt Technologie Deutschland GmbH coupled to the chromatography column. As Curve 1 in FIG. 1 shows, the radius is, depending on the cumulative weight fraction, between 14 nm and about 16 nm and therefore in the size range of the micelles. From the mean micelle weight of about 1.586×10⁶ determined in this way, it can be calculated that each micelle features two molecular aggregates in the core, totalling about 400 molecules of coenzyme Q₁₀ which is enclosed by five Polysorbate 80 molecular aggregates of the same type totalling about 1000 molecules of Polysorbate 80, as schematically illustrated in FIG. 2.

To obtain the water-free phase of 5% by wt. of coenzyme Q₁₀, as much triglyceride and Polysorbate 80 are added while hot (85° C.) to the hot intermediate product such that the mixture comprises about 5 parts of coenzyme Q₁₀, about 16 parts of triglyceride and about 79 parts of Polysorbate 80. For triglyceride, thistle oil is used here which, according to the guiding principles for edible fats and oils of 29/30.11.1983 in the version modified on 2/3.12.1986 (GM Leaflet No. 21, page 379 of 1.8.1987), exhibits a high linoleic acid content of about 67.8% to about 83.2%. Linoleic acid is recommended due to its molecular size which is similar to coenzyme Q₁₀ (the molecular weight of linoleic acid is 725). The mixture is stirred hot until clarity is obtained and then slowly cooled. A water-free phase of coenzyme Q₁₀ is obtained with a particle size, based on the quoted measurements, also in the micelle range. In contrast to the aqueous phase, with the said method a mean micelle diameter of 7.657×10⁵ is measured from which it follows that each micelle in the core exhibits about 200 coenzyme Q₁₀ molecules and five enclosing molecular aggregates totalling 480 molecules of Polysorbate 80, whereby this micelle is surrounded by four other micelles of the same type, of which each exhibits about 190 molecules of thistle oil or linoleic acid and a polysorbate envelope of five molecular aggregates totalling about 480 molecules of polysorbate. A schematic representation of this micelle formation is shown in FIG. 3.

The water-free phase has excellent storage properties and can be dissolved in water at body temperature as required. It is therefore suitable as an additive to nutritional supplements which are normally offered in gelatine capsules. An explanation of the special stability of the water-free phase can in some circumstances be seen in that the central micelle containing the coenzyme Q₁₀ is extensively protected by the four surrounding micelles containing the auxiliary substance, thistle oil, i.e. mainly linoleic acid, particularly from the penetration of polar molecules such as H₂O.

### EXAMPLE 2

### Phytosterin

The starting point is the ADM phytosterol which can be obtained from ADM Nutraceutical, Decatur, Ill. 62526, U.S.A. under the product code 040095. This product contains at least 90% phytosterins and in fact 40%-58% beta-sitosterin, 20%-30% campesterin and 14%-22% stigmasterin as well as up to about 5% each of sitostanol und brassicasterol. In the following this product is briefly designated as phytosterin. Of course, phytosterins from other manufacturers, which contain other compositions in other concentrations, can be treated as described below to give corresponding results. The invention is therefore not restricted to ADM phytosterol.

To produce an aqueous phase, about 320 g of polysorbate, preferably Polysorbate 80 is heated to about 100° C. About 10 g of phytosterin is added to the hot polysorbate and the mixture is stirred, keeping it at a temperature of about 100° C., for about 10 minutes, until a homogeneous and transparent phytosterin concentration of about 3% is produced. The resulting water-free phase, heated where necessary to about 40° C., can be dissolved as required in water at about 20° C.

The said investigation shows that micelles with a molecular aggregate arrangement according to FIG. 4 are present, whereby about 107 phytosterin “molecules” are present in the micelle core and about 207 polysorbate molecules in the Polysorbate 80 envelope. The distribution of the micelle radii can be seen in Curve 3 in FIG. 1 and lies between about 15 nm and about 22 nm.

To produce a water-free phase, first about 100 g of light vegetable oil, for example thistle oil, is heated to about 100° C. About 10 g of phytosterin is added to the hot thistle oil and the mixture stirred, keeping it at a temperature of about 100° C. until, for example after 10 minutes, the phytosterin has completely dissolved. About 220 g of polysorbate, preferably Polysorbate 80, are added to this solution. Stirring is continued at about 100° C. until the mixture becomes transparent with a slight yellow colour. After cooling to room temperature, the water-free phase of a 3% phytosterin concentration is obtained. It is soluble in water and oils and remains stable with respect to hydrochloric and gastric acids (pH<1) and also with respect to heating effects. Measurements show that about 90% of the particles in the water-free phase have a radius of about 16 nm. The molecular aggregate arrangement is shown schematically in FIG. 5, according to which the phytosterin micelle is enclosed by four linoleic acid micelles.

The effect of cholesterol reduction of phytosterins has been known for decades and has also been proven through more recent clinical studies.

### EXAMPLE 3

### ω-3-Fatty Acid

As an example of an ω-3-fatty acid, the product Softgel, which can be obtained from Merck KGaA, Darmstadt, under the product number 1.00743.0200 HI-DHA 25 S. This product contains 25%-28% decosahexaenoic acid (DHA), 5%-8% eicosapentaenoic acid (EPA) and in total about 34%-40% of ω-3-fatty acid. In the following this product is briefly designated as omega-3-fatty acid. Of course, ω-3-fatty acids from other manufacturers can be treated as described in the following, whereby analogous results are obtained.

As an example of the production of an ω-3-fatty acid concentrate, about 800 g of Polysorbate 80 are heated to about 160° C. Then, maintaining the temperature, about 200 g of omega-3-fatty acid is added and stirred for about 5 minutes while maintaining the temperature until a homogeneous mixture is obtained as an intermediate product. The intermediate product produced in this way is transparent and retains its transparency also after slow cooling to room temperature. It can be dissolved as required in water at about 20° C. after brief stirring without turbidity or sedimentation occurring. The intermediate product, which contains about 6% of ω-3-fatty acids, exhibits micelles with a mean radius distribution as given in Curve 4 of FIG. 1, as provided by measurements of the aqueous intermediate product according to the said above method. The mean micelle diameter lies at about 33 nm. The molecular aggregate arrangement is shown FIG. 6; reference is made to the figures entered there.

The clarity and water solubility of the aqueous intermediate product are retained even when gastric acid (hydrochloric acid) is added.

A kilogram of the intermediate product contains about 67 g of ω-3-fatty acids, so that about 3 to 4 g of this intermediate product covers the human daily requirement of ω-3-fatty acids.

### EXAMPLE 4

### Isoflavones

The starting point is a soya bean extract powder which can be obtained from the Archer Daniels Midland Company, U.S.A. under the trade name NOVASOY. This product contains at least 40% by wt. of genistin, daidzin and glycitin and their aglycones in a quantity ratio of 1.3:1.0:0.3. Therefore 100 g of this said extract contain 20.0 g of genistin, 15.4 g daidzin and 4.6 g of glycitin, totalling 40 g of isoflavones. In the following this product is briefly designated as genistin isoflavone. Of course, an appropriate product from another manufacturer can be used provided it contains isoflavones, in a different composition where necessary. For example, a soya bean extract is obtainable from K.-W. Pfannenschmidt GmbH, Hamburg, which contains about 7.58% genistin, 25.43% genistein, 5.48% daidzin and 1.67% daidzein, i.e. about 40% isoflavones. Approximately the same results can be achieved after the treatment described in the following.

About 166 g of the genistin isoflavone is trickled into about 834 g of Polysorbate 80 heated to about 75° C. and the mixture evenly stirred at this temperature for about half an hour. A clear, dark brown intermediate product is obtained without sediment. If about 1-2 ml of this intermediate product is added to ten times that amount of distilled water at room temperature, a clear aqueous phase is obtained. As the measurements show, it contains micelles of which about 96.1% have a radius of about 16 nm to about 20 nm, as illustrated in Curve 5 in FIG. 1.

According to an alternative process, about 100 g of the genistin isoflavone is stirred evenly in about 400 g of water which has previously been heated to about 60° C. The mixture is stirred, maintaining the temperature, for about 10 minutes and then about 500 g of Polysorbate 80 is added with continuation of stirring and during the addition the temperature is increased to about 100° C. The stirring process is continued at this temperature until a clear intermediate product is obtained.

### EXAMPLE 5

### Quercetin

A quercetin dihydrate, which can be obtained from Sigma-Aldrich-Chemie GmbH, Schnelldorf under the article number 83370-100G, can be used as a starting source of quercetin.

67 g of quercetin dihydrate are evenly stirred in about 280 g of water which has been previously heated to about 60° C. The mixture is stirred constantly (using, say, a magnetic stirrer), maintaining the temperature, for about 5 minutes and then during stirring about 653 g of Polysorbate 80 are added, whereby the temperature is increased to approximately 100° C. Stirring continues until a clear, transparent intermediate product containing about 6.7% of quercetin is obtained. An aqueous solution of this product shows micelles of which about 90% exhibit a radius distribution between 17 nm and 19 nm (Curve 6 in FIG. 1).

### EXAMPLE 6

### Lycopene

The starting point is a product which can be obtained from BASE S.A., Switzerland, under the name Tomato Oleoresin. It contains about 40% lycopene and is known in the following as Base lycopene. A product containing lycopene can also be obtained from LycoRed Natural Products Industries Ltd, Beer-Sheba, Israel.

About 100 g of water are heated to just 100° C. and about 50 g of Base lycopene added to the hot water. Maintaining the temperature, the mixture is vigorously stirred for about 5 minutes until it becomes homogeneous and transparent. Then about 850 g of Polysorbate 80 is heated to about 100° C. and added to the mixture. The total mixture that is produced is stirred at about 100° C. until a homogeneous and transparent intermediate product is obtained. After cooling to room or body temperature, the clarity and water solubility of the obtained aqueous phase with a 2% lycopene content are retained. According to Curve 7 in FIG. 1, about 86% of the produced micelles have a radius of about 15 nm to about 16 nm.

Curve 8 in FIG. 1 refers to pure Polysorbate 80 without active substance.

### EXAMPLE 7

### Chondroitin Sulphate

The polymeric galactosamine sulphate, chondroitin, supports the regeneration of overstressed cartilage tissue and reduces symptoms of osteoarthritis.

About 300 g of Polysorbate 80 is added to about 500 g of water heated to about 85° C. and the mixture is stirred at about 85° C. until (after about 5 minutes) the mixture becomes homogeneous and transparent. Then about 200 g of pure chondroitin sulphate are added to this mixture. This mixture is in turn vigorously stirred at the said temperature until a homogeneous and transparent intermediate product is obtained. After cooling to room or body temperature, the clarity and water solubility of the phase so obtained with approximately 20% chondroitin sulphate content are retained.

### EXAMPLE 8

### α-Lipoic Acid

About 870 g of polysorbate, preferably Polysorbate 80, are heated to about 120° C. and then about 130 g of α-lipoic acid—for example the product alpha lipoic acid, Art. No. 1999/010 from K.-W. Pfannenschmidt GmbH, Hamburg—is added and stirred for about 10 minutes maintaining the temperature until a homogeneous, transparent mixture is obtained.

The intermediate product produced in this way can be dissolved as required after cooling with stirring in water at about 25° C. The clarity and water solubility of the water-free phase are retained even when gastric acid (hydrochloric acid) is added to its aqueous solution.

From the above it can be seen that from physiologically effective active substances, which are insoluble or difficult to dissolve in water, a phase of low active substance concentration, soluble in water and oils, can be obtained, which exhibits micelles with radii between about 10 nm and about 20 nm, through treatment with a physiologically compatible solubiliser with an HLB value between 9 and 16, preferably a polysorbate, in particular Polysorbate 80, under heat followed as required by quick cooling to room temperature. This phase is very resistant, primarily to acids such as gastric acid. Above all, it is simpler to deal with than the actual active substances. The resistance of the phase can still be increased in that an auxiliary substance such as linoleic acid is supplemented. As has been shown, the micelles containing the active substance are enclosed with a number of micelles containing the auxiliary substance which provide protection for the active substance micelles.

The micelles formed according to the invention are very stable chemically, microbiologically, mechanically and thermally. They contain proportionally a much larger quantity of mainly lipophilic active substances than comparable liposomes. The active substance concentrates or the formed active substance phases can be added with benefit to foodstuffs, nutritional supplements, skin, hair and dental care products as well as to cosmetic or pharmaceutical media. The active substance concentrates are absolutely stable in gastric acid. Due to the micelle formation the active substances are available to the organism substantially quicker then in active substances administered as emulsions. The resorption in the intestinal region renders participation of the bile acid superfluous.

1. Method for the production of a water-soluble phase of an active substance, which is insoluble or only soluble with difficulty in water and is physiologically effective, characterised in that the active substance is, while heated, stirred with an excess of a physiologically compatible solubiliser with an HLB value between 9 and 16 for the formation of a transparent intermediate product and the intermediate product is cooled to room temperature.

2. Method according to claim 1, characterised in that a polysorbate, especially Polysorbate 80, is used.

3. Method according to claim 1 or 2, characterised in that water is added while heated to the intermediate product.

4. Method according to one of the previous claims, characterised in that an auxiliary substance, for example linoleic acid, is added while heated to the mixture of active substance and solubiliser.

5. Method according to claim 4, characterised in that linoleic acid is used as auxiliary substance.

6. Method according to one of the previous claims, characterised in that the solubiliser is added in such an excess that the intermediate product comprises an active substance concentration of approximately 20% or less.

7. Method according to one of the previous claims, characterised in that the solubiliser is heated to a temperature of about 80° C. to about 100° C.

8. Method according to one of the previous claims, characterised in that the intermediate product is cooled quickly.

9. Method according to one of the previous claims, characterised in that the coenzyme Q₁₀ is used as the active substance.

10. Method according to one of the claims 1 to 8, characterised in that a phytosterin is used as the active substance.

11. Method according to one of the claims 1 to 8, characterised in that an ω-3-fatty acid is used as the active substance.

12. Method according to one of the claims 1 to 8, characterised in that an isoflavone is used as the active substance.

13. Method according to one of the claims 1 to 8, characterised in that quercetin is used as the active substance.

14. Method according to one of the claims 1 to 8, characterised in that lycopene is used as the active substance.

15. Method according to one of the claims 1 to 8, characterised in that chondroitin sulphate is used as the active substance.

16. Water-soluble concentrate of a physiological active substance, insoluble or only soluble with difficulty in water, which comprises an excess of a physiologically compatible solubiliser with an HLB value of 9 to 16.

17. Concentrate according to claim 16, characterised in that the solubiliser is a polysorbate, especially Polysorbate 80.

18. Concentrate according to claim 16 or 17, characterised in that it comprises an auxiliary substance, such as for example linoleic acid.

19. Concentrate according to the claims 16 to 18, characterised in that it exhibits active substance micelles with a mean radius distribution of approximately 10 nm to approximately 20 nm.

20. Concentrate according to claim 19, characterised in that it exhibits auxiliary micelles which enclose the active substance micelles.

21. Concentrate according to claim 19 or 20, characterised in that the active substance micelles contain coenzyme Q₁₀ or a phytosterin or an ω-3-fatty acid or an isoflavone or quercetin or lycopene or chondroitin sulphate or an α-lipoic acid.

22. Application of the concentrate according to the claims 17 to 21 as an additive to foodstuffs, nutritional supplements, to skin, hair and dental care products as well as to cosmetic and/or pharmaceutical media.

23. Concentrate according to one of the claims 16 to 22 characterised by an active substance content of approximately 20% or less.
